(19) United States
(12) Patent Application Publication
(10) Pub. No.: US 2024/0399064 A1
(43) Pub. Date: Dec. 5, 2024

(54) Container Closure with Reverse Coefficient of Thermal Expansion Materials

(71) Applicant: Becton, Dickinson and Company, Franklin Lakes, NJ (US)

(72) Inventors: Nestor Rodriguez San Juan, Hamburg, NJ (US); Leandro Forciniti, Wharton, NJ (US); Gumersindo Rodriguez, Sumter, SC (US)

(73) Assignee: Becton, Dickinson and Company, Franklin Lakes, NJ (US)

(21) Appl. No.: 18/203,127

(22) Filed: May 30, 2023

(51) Int. Cl. A61M 5/31

(52) U.S. Cl. CPC A61M 5/31; A61M 2005/3104; A61M 2005/3106; A61M 2207/00

(57) ABSTRACT

Provided herein is a prefilled syringe including a syringe barrel having a proximal end and a distal end and defining a chamber, the syringe barrel having an opening at the proximal end. The prefilled syringe also includes a plunger assembly inserted through the opening and axially movable within the chamber of the syringe barrel, with the plunger assembly further including a plunger rod having an elongated body extending between a proximal end and a distal end and a stopper attached to the distal end of plunger rod and positioned within the barrel chamber. The stopper is composed of a blended material including an elastomeric material and an intermixed material having a coefficient of thermal expansion lower than the elastomeric material.

### BACKGROUND OF THE INVENTION

### Field of the Invention

The present disclosure relates generally to closure containers such as prefilled syringes and, more particularly, to a sealing component for use with a prefilled syringe that exhibits minimal shrinkage and/or possible expansion at low temperatures, so as to preserve the closure integrity of the syringe liquid content during transport or storage.

### Description of Related Art

Closure containers are used in the medical field for storing and potentially dispensing fluids, such as medications or drugs. The closure container is provided as a stoppered glass or plastic container (e.g., glass or plastic vial or syringe) with an elastomeric closure. The medication or drug stored in the closure container may include, for example, vaccines or cell therapies.

When a closure container is provided as a prefilled syringe for dispensing fluids, the syringe will typically include a syringe barrel with an opening at a distal end and a plunger assembly inserted through the opposite proximal end of the barrel. The plunger assembly typically includes an elongated plunger rod extending out of the barrel, and a plunger head or stopper disposed at the distal end of the plunger rod. The stopper or plunger stopper is typically made of elastomeric material, and comprises a body with a tail disposed at its proximal end adapted for attachment to the distal end of the plunger rod, and a head disposed at its distal end. On an external cylindrical wall of the body is defined a plurality of annular outwardly protruding ribs adapted to ensure the container closure integrity of a syringe when the stopper is inserted into the syringe.

After a period of transport and/or storage, during delivery of the medication to a patient, the opening of the syringe barrel on the distal end is adapted for fluid communication with a patient, such as through a needle already attached to the syringe barrel (stacked needle syringe barrel), or through a hypodermic needle fitted at the distal end of the syringe barrel (Luer syringe barrel), or through a Luer-type fitting (needleless access device) fitted at the distal end of the syringe barrel and connected to a fluid line of a patient. Upon the user applying a force to move the plunger assembly through the syringe barrel towards the distal end of the syringe barrel, the content of the syringe is thereby pulled out of the syringe barrel through the opening at the distal end for delivery to the patient. Such an operation is well known in the medical field, and medical practitioners have become well accustomed to the use of such common fluid delivery procedures using prefilled syringes.

Prefilled syringes provide the convenience of rapidly delivering the content to a patient without the need to first aspirate the medication from another container and meter its volume. At the same time, there is a need to maintain sterility and container closure integrity of the medication inside the prefilled syringe, including over an extended period of time, and throughout exposure to various storage and transport conditions. In some circumstances, prefilled syringes are subjected to extremely cold temperatures—including temperatures down to as low as −80 to −196° C. (i.e., liquid nitrogen temperature)—in order to properly store the medication contained therein (e.g., a vaccine or cell therapy). In other circumstances, prefilled syringes are subjected to a combination of cold temperatures and higher pressures, such as when being transported on a plane, for example. Maintaining container closure integrity in these conditions is a big challenge for prefilled syringes based, at least in part, on the coefficient of thermal expansion (CTE) mismatch between the stopper and the barrel materials. This CTE mismatch is even more pronounced when the barrel of the prefilled syringe is formed of glass, with it known that a glass substrate (i.e., the barrel) typically has a CTE_{g}=5 ppm/° C., while rubber such as bromobutyl (i.e., the stopper) has a CTE_{S}=200 ppm/° C. at room temperature (T_amb). Assuming constant CTEs from room temperature to deep cold conditions, this level of CTE mismatch may result in an interference rate between the stopper and the barrel dropping by approximately 30%—this being due at least in part to the elastomeric stopper being brought to its glass transition temperature (Tg) where the elastic properties of the stopper are altered so that it becomes “glass-like” and loses its ability to apply a strong contact pressure against the barrel and maintain a seal therewith. Any contact pressure variation/reduction between the stopper and barrel during the thermal cycle may present risk on the container closure integrity either by creating a leaking channel (partial lack of contact) at the barrel-stopper interface and/or by enabling stopper movement inside the barrel during the freeze-thaw cycles.

A large CTE mismatch between the barrel and the stopper can also cause issues with subsequent container closure integrity when initially inserting the stopper into the barrel. That is, during initial assembly of a pre-filled syringe, a stopper is typically placed by using vent tube stoppering, which consists in compressing the stopper (using a vent tube stoppering tool) to have a diameter significantly smaller than the barrel internal diameter, before placing the stopper into the barrel and allowing the stopper to expand back and become sealed in the barrel. For many stopper materials, including where a rubber stopper is coated with PTFE, this can lead to damage to the rubber material and/or the coating thereon, which may lead to compromised container closure integrity and/or particle generation.

Accordingly, a need exists in the art for closure containers, including prefilled syringes, that are able to maintain sterility and container closure integrity even at extremely low cold storage temperatures. A need further exists for a method of manufacturing such closure containers that maintains container closure integrity throughout and subsequent to such manufacturing.

### SUMMARY OF THE INVENTION

Provided herein is a prefilled syringe including a syringe barrel having a proximal end and a distal end and defining a chamber, the syringe barrel having an opening at the proximal end. The prefilled syringe also includes a plunger assembly inserted through the opening and axially movable within the chamber of the syringe barrel, with the plunger assembly further including a plunger rod having an elongated body extending between a proximal end and a distal end and a stopper attached to the distal end of plunger rod and positioned within the barrel chamber. The stopper is composed of a blended material including an elastomeric material and an intermixed material having a coefficient of thermal expansion lower than the elastomeric material.

In certain configurations, the intermixed material has a negative coefficient of thermal expansion.

In certain configurations, the blended material of the stopper has a coefficient of thermal expansion matching a coefficient of thermal expansion of the syringe barrel or lower than the coefficient of thermal expansion of the syringe barrel.

In certain configurations, the stopper is configured to expand at room temperature or at cold storage temperatures of 0° C. and lower, so as to maintain a closure integrity between the stopper and the syringe barrel.

In certain configurations, the intermixed material is graphene, a hexagonal boron nitride compound (coronene), ScF3 compound, ZrW2O8, or beta-eucryptite and Ca2RuO4.

In certain configurations, the stopper includes an inert coating applied to at least a portion of an outer surface thereof.

In certain configurations, the inert coating is a parylene coating.

In certain configurations, the syringe barrel is composed of a glass substrate.

In certain configurations, the syringe barrel is composed of a polymeric substrate.

In certain configurations, the stopper is a molded stopper formed via molding of a blend of the elastomeric material and the intermixed material.

In certain configurations, the syringe includes a distal end cap positioned over a nozzle at the distal end of the barrel chamber, wherein the distal end cap is composed of a blended material including a polymeric material and an intermixed material having a coefficient of thermal expansion lower than the polymeric material.

Also provided herein is an apparatus for storing and/or delivering a fluid. The apparatus includes a container having a proximal end and a distal end and defining a chamber, with the container having an opening at the proximal end. The apparatus also includes a sealing component positioned at least partially within the chamber such that at least a portion of the sealing component forms an interference fit with the container, wherein the sealing component is composed of a blended material including an elastomeric material and an intermixed material having a coefficient of thermal expansion lower than the elastomeric material.

In certain configurations, the intermixed material has a negative coefficient of thermal expansion (NCTE).

In certain configurations, the intermixed material is graphene, hexagonal boron nitride (coronene) compound, ScF3 compound, ZrW2O8, or beta-eucryptite and Ca2RuO4.

In certain configurations, the stopper includes an inert coating applied to at least a portion of an outer surface thereof.

In certain configurations, the container is composed of one of a glass substrate and a polymeric substrate.

In certain configurations, the apparatus is a prefilled syringe, with the container being a syringe barrel and the scaling component being a stopper affixed to a distal end of a plunger rod.

In certain configurations, the apparatus includes a distal end cap positioned over a nozzle at the distal end of the chamber, and wherein the distal end cap comprises a blended material including another polymeric material and an intermixed material having a coefficient of thermal expansion lower than the polymeric material.

Also provided herein is a method of assembling a prefilled syringe. The method includes providing a syringe barrel comprising a proximal end and a distal end and defining a chamber, the syringe barrel having an opening at the proximal end. The method also includes providing a plunger assembly insertable into the opening, the plunger assembly including a plunger rod comprising an elongated body and extending between a proximal end and a distal end and a stopper attached to the distal end of plunger rod and positioned within the barrel chamber, wherein the stopper comprises a blended material including an elastomeric material and an intermixed material having a negative coefficient of thermal expansion that is lower than a coefficient of thermal expansion the elastomeric material. The method further includes heating a vent tube stoppering tool that holds the plunger assembly therein, with the vent tube stoppering tool heated to a temperature that causes the stopper to contract, due to the intermixed material with the negative coefficient of thermal expansion, and inserting the plunger assembly into the syringe barrel using the vent tube stoppering tool.

In certain configurations, the method also includes filling the barrel with a liquid solution prior to inserting of the plunger assembly.

### BRIEF DESCRIPTION OF THE DRAWINGS

FIG. 1 is a perspective view of a prefilled syringe, according to a non-limiting embodiment described herein;

FIG. 2 is an exploded view of the syringe of FIG. 1;

FIG. 3 is a side cross-sectional view of the syringe of FIG. 1, with a plunger assembly thereof in an initial or pre-use position;

FIG. 4 is a detailed view of a stopper positioned within a barrel of the syringe of FIG. 1; and

FIG. 5 is a cross-sectional view of a vial and associated sealing component, according to another non-limiting embodiment described herein.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”. “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

In the present disclosure, the distal end of a component or of a device means the end furthest away from the hand of the user and the proximal end means the end closest to the hand of the user, when the component or device is in the use position, i.e., when the user is holding a syringe in preparation for or during use. Similarly, in this application, the terms “in the distal direction” and “distally” mean in the direction toward the distal tip of the syringe, and the terms “in the proximal direction” and “proximally” mean in the direction opposite the direction of the distal tip of the syringe.

Aspects and embodiments of the disclosure are directed to the use of low or negative coefficient of thermal expansion (NCTE) materials in a container closure or sealing component. The use of the low or negative CTE material allows for a sealing component to have a CTE more closely matched to the CTE of the container in which it is seated. In embodiments where the scaling component has a negative CTE, the sealing component may be configured to expand when a container is exposed to cold storage temperatures.

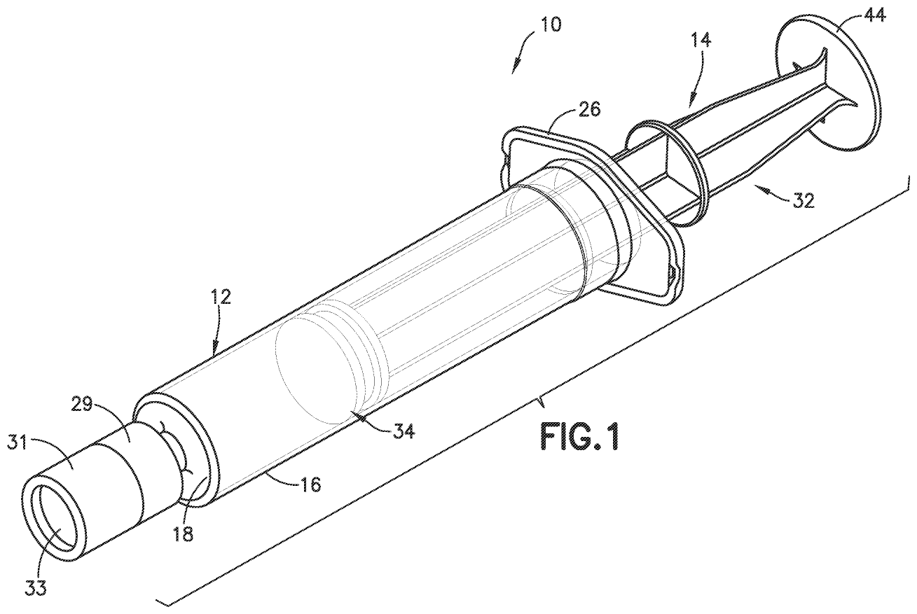

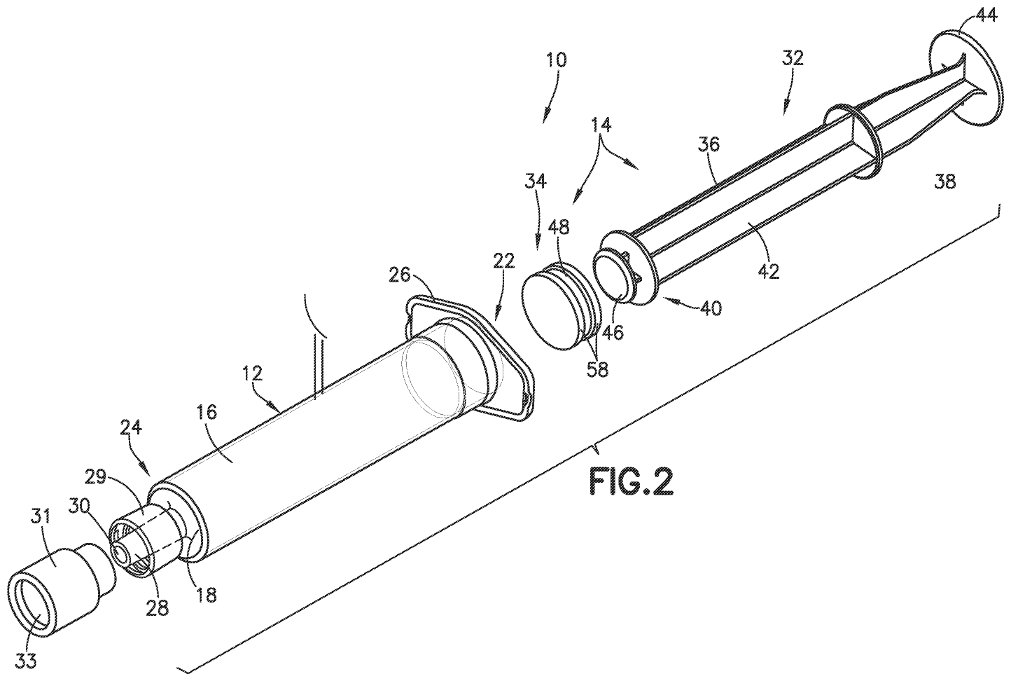

Referring to FIGS. 1 and 2, shown is a non-limiting embodiment of a prefilled syringe 10 with which aspects or embodiments of the disclosure may be implemented. The syringe 10 generally includes a syringe barrel 12 and a plunger assembly 14. The plunger assembly 14 is axially movable within the syringe barrel 12 to an advanced position to facilitate administering of an injectable fluid (e.g., medication) to a patient, for example.

The syringe barrel 12 is formed of a generally cylindrical outer wall 16 and an end wall 18 that collectively define a chamber 20 for retaining fluid therein. The syringe barrel 12 includes an open proximal end 22 configured to receive the plunger assembly 14 therein and a distal end 24 at which end wall 18 is positioned. The proximal end 22 of the syringe barrel 12 may include a flange 26 to facilitate handling and positioning of the syringe 10 and to maintain the relative position of the syringe barrel 12 with respect to the plunger assembly 14 during medication administration. In one embodiment, the distal end 24 of syringe barrel 12 may include a tip 28 that extends distally outward from the end wall 18 and defines a lumen 30 in fluid communication with the chamber 20. A luer lock adapter 29 is mounted on an external surface of the tip 28 that is coupleable to a rigid cap 31 that covers the tip 28 and/or to a corresponding needle hub (not shown) to which the syringe 10 is to be brought into engagement for administering fluid to a patient. The adapter 29 comprises a cylindrical body having a threaded inner surface that is configured to engage an outer surface of a threaded portion of the rigid cap 31. It can be appreciated that, in other embodiments, syringe 10 may be provided as a staked needle syringe, where a needle is secured to syringe barrel 12 and extends out distally therefrom, with a needle shield positioned over the needle.

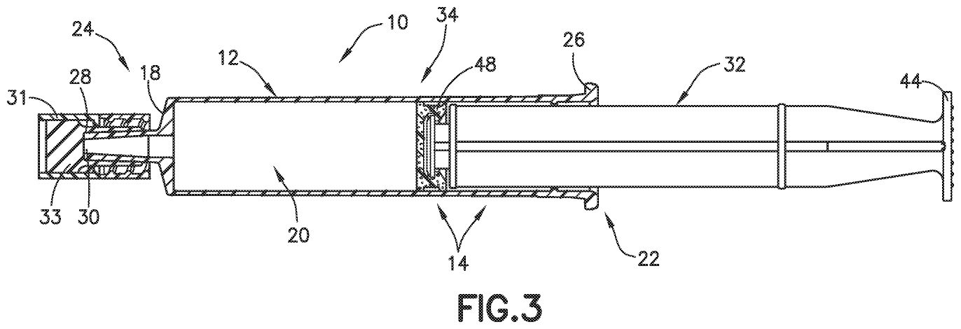

As best shown in FIG. 3, the rigid cap 31 may be configured as a rigid cap that mates with the adapter 29, so as to provide a fluid-tight seal over the tip 28, with a proximal end of the rigid cap 31 configured to engage the adapter 29 and a distal end configured as a closed end, with some embodiments of rigid cap 31 including an elastomeric inner cap 33 (“RiTC”) positioned in the rigid cap 31 so as to cover the tip 28 and be partially inserted into lumen 30 of tip 28.

The plunger assembly 14 of syringe 10 is formed of an elongate plunger rod 32 (more generally “plunger 32,” as used hereafter) and a plunger head or stopper 34. The plunger 32 may include a main body 36 extending between a plunger proximal end 38 and a plunger distal end 40. In some embodiments, the main body 36 may include a plurality of elongate vanes or walls 42 extending axially along a length thereof between the plunger proximal end 38 and the plunger distal end 40. A thumb press 44 is positioned at the plunger proximal end 38 that may be engaged by a thumb (or other finger) of the user to apply a distally directed force to the plunger assembly 14 to move the plunger 32 with respect to the syringe barrel 12. In some embodiments, a flanged extension member 46 (e.g., disc-shaped flange) is positioned at the plunger distal end 40 that is configured to mate with the stopper 34. In other embodiments, the plunger distal end 40 may include a female receptacle formed therein that is configured to receive and connect to a protrusion (e.g., pin) extending out proximally from the stopper, with the protrusion and receptacle engaging via threading, for example.

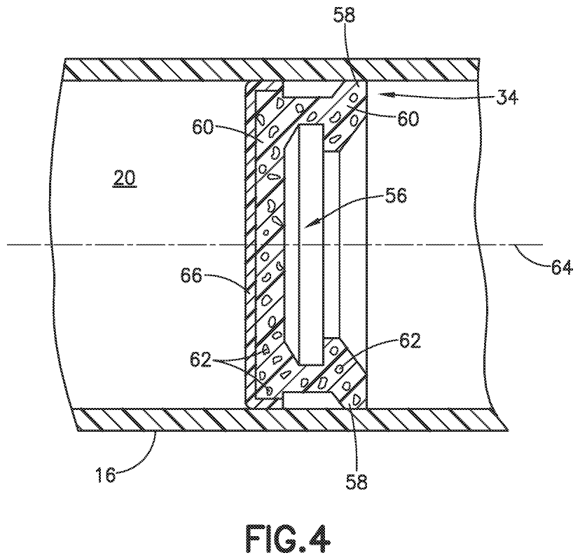

The stopper 34 of plunger assembly 14 is positioned at the plunger distal end 40 so as to be movable along with the plunger 32 within the chamber 20 of syringe barrel 12. The stopper 34 may be made from a material that is different from the material of the plunger 32 and that is capable of forming a tight seal with the syringe barrel 12 as it is advanced therethrough. In some embodiments, the stopper 34 includes a receptacle 56 (FIG. 4) formed therein that is sized and configured to receive the flanged extension member 46 of plunger 32, with the flanged extension member 46 coupling with the receptacle 56 via a press fit connection, for example, to secure the stopper 34 to the plunger 32, although it can be appreciated that the attachment member 48 and plunger distal end 40 can be secured by other techniques known in the art. The attachment member 48 may further include a pair of spaced-apart annular flanges 58 formed thereon (i.e., O-rings) that form a tight seal with the cylindrical outer wall 16 of syringe barrel 12.

As indicated above, syringe 10 may be provided as a prefilled syringe, such that the syringe 10 contains a drug or medication prefilled within chamber 20. In some embodiments, the drug or medication within the chamber 20 of prefilled syringe 10 may be a drug or medication that requires cold storage (i.e., cryogenic storage) at temperatures down to as low as −80 to −196° C. In order to maintain container closure integrity at these deep cold storage temperatures, the syringe 10 may thus be specifically configured to maintain sterility and container closure integrity at cold storage temperatures. According to aspects of the disclosure, the stopper 34 is composed of desired materials that minimize a CTE mismatch between the stopper 34 and the barrel materials and that allows that stopper 34 to maintain some degree of contact pressure at cold storage temperatures. That is, according to aspects or embodiments of the disclosure, the syringe barrel 12 may be formed of glass having a coefficient of thermal expansion of CTE_g=5 ppm/° C. or of a polymeric material having a coefficient of thermal expansion of CTE_p=50 ppm/° C., while the stopper 34 may be formed of a mixture of an elastomeric material and other low or negative CTE material that provides an overall CTE of the stopper 34 that is close to or below the CTE of the barrel material—i.e., that matches the CTE of the barrel material or is below the CTE of the barrel material—so as to provide an improvement over a typical elastomeric stopper 34 with a high CTE, such as bromobutyl with a CTE_S=200 ppm/° C. at room temperature. By more closely matching the CTE of the stopper 34 to the CTE of the barrel 12 or making the CTE of the stopper 34 less than the CTE of the barrel 12, an interference rate between the stopper 34 and the barrel 12 may be maintained even down at extremely low cold storage temperatures where an elastomeric material of the stopper 34 is brought to its glass transition temperature (Tg).

According to aspects or embodiments of the disclosure, and as shown in FIG. 4, the stopper 34 is formed of a flexible, elastomeric material 60 mixed with another (intermixed) material 62 having a CTE that is less than the CTE of the elastomeric material and, in some embodiments, has a negative CTE at the storage temperature, which may be below 0° C. and down to as low as −80 to −196° C. The intermixed material 62 is preferably interspersed with the elastomeric material 60 in a manner that will counter contraction of the elastomeric material 60 when the elastomer is near or goes below the Tg of the elastomer, thus allowing for the stopper 34 to maintain contact pressure with the barrel 12. For example, the syringe 10 may have a centerline axis 64, wherein the barrel 12 and stopper 34 are coaxial along the centerline axis 64 when assembled, and when the syringe 10 is stored at low temperatures, such as cryogenic temperatures, the elastomeric material 60 of the stopper 34 may contract radially towards the centerline axis 64; however, the intermixed material 62 having a lower coefficient of thermal expansion preferably contracts less, or more preferably, may expand radially away from centerline axis 64 if the CTE of the intermixed material 62 is negative at the storage temperature.

The amount of the intermixed material 62 incorporated into the stopper 34 is dependent on the difference between the CTEs of the intermixed material 62 and elastomeric material 60 and/or on the CTE of the substrate material from which the barrel 12 is formed (e.g., glass or a polymeric material). For example, if the intermixed material 62 has a CTE that is much less than the CTE of the elastomeric material 60, then more of the intermixed material 62 may be required to attain the desired counter-action of the expected contraction of the elastomeric material 60 and/or to achieve a negative CTE for the overall stopper 34. However, if the difference between the CTEs between the two materials is small, a lesser amount of the intermixed material 62 may be required to achieve the same degree of resistance to the contraction of the elastomeric material 60 and/or to configure the stopper 34 to expand at a low temperature. Additionally, if the barrel 12 is formed of glass and has a very low CTE (i.e., CTE_g=5 ppm/° C.), then a greater amount of the intermixed material 62 may be required to bring the total CTE of the stopper 34 closer to the CTE of the barrel 12 (or below the CTE of the barrel 12), while if the barrel 12 is formed of a polymeric material and has a very higher CTE (i.e., CTE_g=50 ppm/° C.), then a smaller amount of the intermixed material 62 may be required to bring the total CTE of the stopper 34 closer to the CTE of the barrel 12 (or below the CTE of the barrel 12).

Examples of the elastomeric material 60 for forming the stepper according to the various embodiments of the present disclosure include, but are not limited to, synthetic or natural rubbers, such as butyl rubber, isoprene rubber, butadiene rubber, halogenated butyl rubber (e.g., bromobutyl rubber), ethylene propylene terpolymer, silicone rubber; combinations thereof and the like. Preferably, the elastomeric material 60 is a butyl or halobutyl elastomer. The elastomeric material 60 may further comprise one or more additives such as a vulcanizing agent, a vulcanizing accelerator, a vulcanizing activator, a processing aid, a filler, and a reinforcing agent to improve or enhance the properties of the elastomeric material 60.

Examples of the intermixed material 62 included in the stopper 34 according to the various embodiments of the present disclosure include, but are not limited to, graphene, hexagonal boron nitride (coronene) compounds, ScF3, ceramic types such as zirconium ZrW2O8, or beta-eucryptite and Ca2RuO4, perovskite BiNiO3, and nano metal oxides like CuO.

In accordance with one non-limiting aspect or embodiment of the disclosure, the stopper 34 is formed of a rubber-graphene blend, with a 0.1 to 19% graphene content. The amount of graphene or other NCTE materials may replace partially or totally typical rubber reinforcement materials like clays or silica. In accordance with another embodiment under this approach, the stopper 34 could be a rubber blend with up to 40% of any of the NCTE compounds described above.

According to aspects or embodiments of the disclosure, the stopper 34 may be manufactured via a molding process, with the elastomeric material 60 and intermixed material 62 being blended together prior to molding. Upon blending of the materials, the stopper 34 may be molded under relative high temperatures (e.g., 120-190° C.) to allow the cross linking of the molecules therein and obtaining of the elastic and resilience properties of the stopper 34. With the inclusion of an intermixed material 62, such as graphene, the stopper 34 may be molded while avoiding scorching of the elastomeric material 60 therein, with the graphene providing improved functional performance of the stopper 34 by increasing uniformity of the rubber curing state and toughness, and lowering the CTE of the stopper 34, as previously described.

According to some aspects or embodiments of the disclosure, the stopper 34 may also include a protective coating 66 on at least a portion thereof that prevents potential interaction between the intermixed material 62 with the drug or medication contained within the chamber 20 of barrel 12. The coating 66 preferably covers at least a portion of the outer surface of the stopper 34 that is most likely to contact the drug or medication within the barrel 12 and prevents the leaching of the intermixed material 62 into the drug or medication. While coating 66 is shown in FIG. 4 as being applied to only a bottom portion of stopper 34, the coating 66 may be to an entirety of the outer surface of the stopper 34, according to other embodiments. In a preferred embodiment, the coating 66 is an inert film, preferably a parylene or fluoropolymer coating. According to one embodiment, the coating is a parylene C coating.

According to aspects or embodiments of the disclosure, it is recognized that when the intermixed material 62 selected to prevent the overall contraction of the stopper 34 at low temperatures (i.e., cryogenic temperatures) has a negative CTE, the intermixed material 62 may potentially jeopardize the container closure integrity if the syringe 10 is exposed to higher temperatures. That is, as a negative CTE material shrinks at higher temperatures, the interference rate between the stopper 34 and the barrel 12 may drop below an acceptable amount if the temperature to which the stopper 34 is exposed becomes unacceptably high (e.g., exceeds 25° C.), such that a leaking channel may form at the barrel-stopper interface and/or the stopper 34 may move inside the barrel 12. Accordingly, when blending a negative CTE intermixed material 62 with the elastomeric material 60, a maximum high temperature should be defined at which the syringe 10 should be kept below.

According to embodiments of the disclosure, it is desirable for the stopper 34 to be designed such that the interference between the stopper 34 and the barrel 12 does not drop by more than 30% of the initial interference, which may be an interference of 0.35 mm as an example, as this would potentially jeopardize the container closure integrity. The temperature at which interference drops by 30%, T_{30%}, may be defined by:

$${T}_{30 \%} = {T}_{a m b} - 0.3 * \frac{{S}_{OD} - {B}_{ID}}{{S}_{OD} * {CTE}_{S} - {B}_{ID} * {CTE}_{g}}$$

where T_{amb} is the ambient temperature, S_{OD} is the stopper outer diameter, B_{ID} is the barrel inner diameter, CTE_{S} is the CTE of the stopper, and CTE_{g} is the CTE of the (glass) barrel, with the above relationship assuming that the CTE is constant over an entire temperature range.

Using the above definition for T_{30%}, a relationship for interaction of various stoppers (i.e., stoppers with various CTEs, including negative CTEs where the stopper 34 shrinks at high temperatures) with a glass barrel substrate or a plastic barrel substrate may be provided, as illustrated in Table 1 provided here below, with the following example assuming an interference of 0.35 mm between the stopper 34 and barrel 12 (e.g. stopper outer diameter P_{OD}=6.7 mm, barrel inner diameter B_{ID}=6.35 mm). Table 1 thus illustrates minimum/maximum temperatures at which T_{30%} is reached.

TABLE 1

| | T_{30%} w/ Glass | T_{30%} w/ Plastic |
|---|---|---|
| CTE_{S} | barrel (° C. or K) | barrel (° C. or K) |
| 2.00E−04 | −76.64 | −104.50 |
| 1.50E−04 | −111.61 | −167.17 |
| 1.00E−04 | −183.25 | 0 Kelvin |
| 5.00E−05 | 0 Kelvin | 0 Kelvin |
| 5.00E−06 | 0 Kelvin | 501.19 |
| 0.00E+00 | 4276.97 | 450.20 |
| −5.00E−06 | 2078.23 | 409.07 |
| −5.00E−05 | 388.15 | 230.32 |
| −1.00E−04 | 214.67 | 160.34 |
| −1.50E−04 | 153.36 | 125.93 |
| −2.00E−04 | 122.00 | 105.48 |

It is recognized that the above example is purely illustrative, as the temperature calculated will also depend on the product design (barrel ID, stopper OD, CTE over the temperature range, etc.):

While it is recognized that the exposing of stopper 34 to high temperatures during storage/transport can cause the interference rate between the stopper 34 and the barrel 12 to drop below an acceptable amount when the stopper 34 includes an intermixed material 62 with a negative CTE-thereby jeopardizing container closure integrity—it is further recognized that such high temperatures may be beneficial during initial assembly of a container or syringe 10. That is, a stopper 34 that includes an intermixed material 62 with a negative CTE can present advantages during assembly due to the dimensions of the stopper 34 shrinking at temperatures above room temperature. In inserting the stopper 34 into a syringe 10, a vent tube stoppering tool used to insert the stopper 34 can be heated to facilitate the stopper insertion process, as heating of the tool causes dimensions of the stopper 34 to shrink, thus reducing the interference rate between the stopper 34 and vent tube stoppering tool-which lessens the chances of the stopper 34 (and/or a coating 66 thereon) becoming damaged during assembly, thereby ensuring a subsequent container closure integrity when the stopper 34 is inserted in the barrel 12.

Therefore, according to aspects or embodiments of the disclosure, a method of assembling a prefilled syringe may be provided that includes providing a syringe barrel, providing a plunger assembly insertable into the syringe barrel and that includes a stopper comprising a blended material including an elastomeric material and an intermixed material having a negative coefficient of thermal expansion that is lower than a coefficient of thermal expansion of the elastomeric material, heating the vent tube stoppering tool that holds the plunger assembly therein, with the vent tube stoppering tool heated to a temperature that causes the stopper to contract (e.g., above 25° C.), and inserting the plunger assembly into the syringe barrel using the vent tube stoppering tool. The method can also include filling the barrel with a liquid solution prior to inserting of the plunger assembly.

According to aspects or embodiments of the disclosure, in addition to the stopper 34 including a material therein having a low or negative CTE, portions of the rigid cap 31 (or of a needle shield) may also be composed in part of a low or negative CTE material. In some embodiments, the inner cap 33 (or a rubber inner portion of a needle shield) may be formed of a blended material including an elastomeric material and intermixed material having a negative coefficient of thermal expansion that is lower than a coefficient of thermal expansion of the elastomeric material, such as previously described for stopper 34. The inner cap 33 may therefore be specifically configured to have a CTE that closely matches that of barrel 12 (i.e., of tip 28 of the barrel), such that closure of the tip 28 is ensured and the closure integrity of the syringe 10 maintained.

In still other embodiments, other portions of rigid cap 31 (or a needle shield) may be formed of a blend of a rigid polymeric material and an intermixed material 62 having a low or negative CTE. The cap portion may be formed as a graphene based polymer nanocomposite, with graphene being mixed with any suitable polymeric material, including polypropylene or polyethylene, as non-limiting examples. The polymeric portion of the rigid cap 31 may therefore also be specifically configured to have a CTE that closely matches that of a glass barrel 12 (i.e., tip 28).

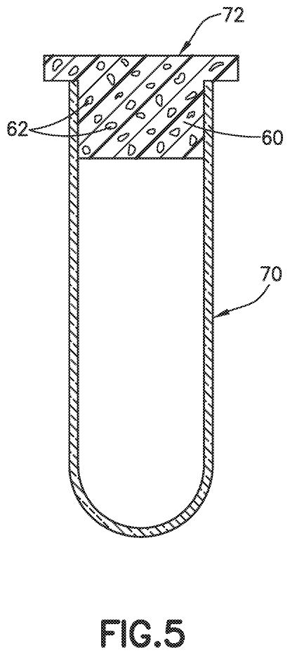

While aspects and embodiments of the disclosure described above are directed to a prefilled syringe 10 including a barrel 12 and plunger assembly 14 having a stopper 34, it is recognized that additional embodiments of the disclosure may be directed to other apparatuses—i.e., to other containers and sealing elements-including a vial and an associated vial stopper, for example. FIG. 5 generally illustrates such an apparatus that includes a container (vial) 70 and a scaling element (vial stopper) 72, where the vial 70 may be constructed of glass or a polymeric material and the vial stopper 72 may be formed of a blend of elastomeric material and a material with a low or negative CTE, as described in detail above regarding the syringe 10 of FIGS. 1-4.

According to embodiments, a negative CTE material 62 such as graphene may be blended into an elastomeric material 60 to form the vial stopper 72, so as to counter contraction of the vial stopper 72. That is, when the vial 70 and vial stopper 72 are stored at low temperatures, such as cryogenic temperatures, the elastomeric material 60 of the vial stopper 72 may contract radially inward and away from vial 70; however, the intermixed material 62 having a lower coefficient of thermal expansion preferably contracts less, or more preferably, may expand radially outward toward vial 70 if the CTE of the intermixed material 62 is negative at the storage temperature.

Beneficially, embodiments of the invention thus provide a container closure or scaling component that includes low or negative coefficient of thermal expansion (CTE) materials intermixed with another material, such as an elastomeric material. The use of the low or negative CTE material allows for a sealing component to have a CTE more closely matched to the CTE of the container in which it is seated. In embodiments where the sealing component has a negative CTE, the sealing component may be configured to expand when a container is exposed to cold storage temperatures, thereby further ensuring container closure integrity even as extreme low cold storage temperatures.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. A prefilled syringe, comprising:
a syringe barrel comprising a proximal end and a distal end and defining a chamber, the syringe barrel having an opening at the proximal end; and
a plunger assembly inserted through the opening and axially movable within the chamber of the syringe barrel, the plunger assembly including:
a plunger rod comprising an elongated body and extending between a proximal end and a distal end; and
a stopper attached to the distal end of plunger rod and positioned within the barrel chamber;
wherein the stopper comprises a blended material including an elastomeric material and an intermixed material having a coefficient of thermal expansion lower than the elastomeric material.

2. The syringe of claim 1, wherein the intermixed material has a negative coefficient of thermal expansion.

3. The syringe of claim 2, wherein the blended material of the stopper has a coefficient of thermal expansion matching a coefficient of thermal expansion of the syringe barrel or lower than the coefficient of thermal expansion of the syringe barrel.

4. The syringe of claim 2, wherein the stopper is configured to expand at room temperature or at cold storage temperatures of 0° C. and lower, so as to maintain a closure integrity between the stopper and the syringe barrel.

5. The syringe of claim 2, wherein the intermixed material comprises graphene, a hexagonal boron nitride (coronene) compound, ScF3 compound, ZrW2O8, or beta-eucryptite and Ca2RuO4.

6. The syringe of claim 1, wherein the stopper comprises an inert coating applied to at least a portion of an outer surface thereof.

7. The syringe of claim 6, wherein the inert coating comprises a parylene coating.

8. The syringe of claim 1, wherein the syringe barrel is composed of a glass substrate.

9. The syringe of claim 1, wherein the syringe barrel is composed of a polymeric substrate.

10. The syringe of claim 1, wherein the stopper comprises a molded stopper formed via molding of a blend of the elastomeric material and the intermixed material.

11. The syringe of claim 1, further comprising a distal end cap positioned over a nozzle at the distal end of the barrel chamber, and wherein the distal end cap comprises a blended material including a polymeric material and an intermixed material having a coefficient of thermal expansion lower than the polymeric material.

12. An apparatus for storing and/or delivering a fluid, the apparatus comprising:
a container having a proximal end and a distal end and defining a chamber, the container having an opening at the proximal end; and
a sealing component positioned at least partially within the chamber such that at least a portion of the sealing component forms an interference fit with the container;
wherein the sealing component comprises a blended material including an elastomeric material and an intermixed material having a coefficient of thermal expansion lower than the elastomeric material.

13. The apparatus of claim 12, wherein the intermixed material has a negative coefficient of thermal expansion.

14. The apparatus of claim 13, wherein the intermixed material comprises graphene, hexagonal boron nitride (coronene) compound, ScF3 compound, ZrW2O8, or beta-eucryptite and Ca2RuO4.

15. The apparatus of claim 12, wherein the stopper comprises an inert coating applied to at least a portion of an outer surface thereof.

16. The apparatus of claim 12, wherein the container is composed of a glass substrate or a polymeric substrate.

17. The apparatus of claim 12, wherein the apparatus is a prefilled syringe, with the container comprising a syringe barrel and the sealing component comprising a stopper affixed to a distal end of a plunger rod.

18. The apparatus of claim 17, further comprising a distal end cap positioned over a nozzle at the distal end of the chamber, and wherein the distal end cap comprises a blended material including another polymeric material and an intermixed material having a coefficient of thermal expansion lower than the polymeric material.

19. A method of assembling a prefilled syringe, the method comprising:
providing a syringe barrel comprising a proximal end and a distal end and defining a chamber, the syringe barrel having an opening at the proximal end;
providing a plunger assembly insertable into the opening, the plunger assembly including:
a plunger rod comprising an elongated body and extending between a proximal end and a distal end; and
a stopper attached to the distal end of plunger rod and positioned within the barrel chamber;
wherein the stopper comprises a blended material including an elastomeric material and an intermixed material having a negative coefficient of thermal expansion that is lower than a coefficient of thermal expansion the elastomeric material;
heating a vent tube stoppering tool that holds the plunger assembly therein, with the vent tube stoppering tool heated to a temperature that causes the stopper to contract, due to the intermixed material with the negative coefficient of thermal expansion; and
inserting the plunger assembly into the syringe barrel using the vent tube stoppering tool.

20. The method of claim 19, further comprising filling the barrel with a liquid solution prior to inserting of the plunger assembly.
